(19) United States
(12) Patent Application Publication
(10) Pub. No.: US 2026/0257912 A1
(43) Pub. Date: Sep. 3, 2026

(54) ANAEROBIC DIGESTER-BASED SYSTEM FOR RNG AND HYDROGEN PRODUCTIONS

(71) Applicant: University of Southern California, Los Angeles, CA (US)

(72) Inventors: Theodore T. TSOTSIS, Huntington Beach, CA (US); Razeih ETEZADI, Los Angeles, CA (US)

(73) Assignee: University of Southern California, Los Angeles, CA (US)

(21) Appl. No.: 19/535,208

(22) Filed: Feb. 10, 2026

Related U.S. Application Data
(60) Provisional application No. 63/756,615, filed on Feb. 10, 2025.

(51) Int. Cl. C01B 3/26; C01B 3/56; C07C 1/12

(52) U.S. Cl. CPC C01B 3/26; C01B 3/56; C07C 1/12; C01B 2203/0233; C01B 2203/0283; C01B 2203/062; C01B 2203/085; C01B 2203/0877; C01B 2203/148; C01B 2203/1642; C01B 2203/82; C01B 2210/0014; C01B 2210/0051

(57) ABSTRACT

An integrated waste-to-energy system converts organic waste into hydrogen and renewable natural gas (RNG). The system includes an anaerobic digester producing biogas, a gas cleaning system, a catalytic reforming system (CRS) that converts the cleaned biogas into hydrogen-rich syngas, and a hydrogen separation unit producing ultrapure hydrogen. The CRS includes steam generation, steam-biogas mixing, an electrically heated steam methane reforming reactor, and a water gas shift reactor, with waste heat recuperation for steam production. A hydrogen-lean off-gas stream is recycled to the anaerobic digester for biomethanation to generate RNG. A dual recirculation architecture enables closed-loop carbon recycling and selective operation in hydrogen production or biomethane enhancement modes. The system may be modular, containerized, and digitally optimized in real time.

### CROSS-REFERENCE TO RELATED APPLICATIONS

This application claims the benefit of U.S. provisional application Ser. No. 63/756,615 filed Feb. 10, 2025, the disclosure of which is hereby incorporated in its entirety by reference herein.

### FIELD OF THE DISCLOSURE

The subject disclosure relates generally to an integrated waste-to-energy conversion system.

### BACKGROUND

Anaerobic digestion is a well-known biological process in which microorganisms break down organic waste materials, such as municipal solid waste, agricultural residues, and industrial food waste, to produce biogas comprising primarily methane and carbon dioxide. While anaerobic digestion provides a useful means of waste management and biogas production, the resulting biogas is a relatively low-value energy product that typically requires significant upgrading or further processing to be suitable for high-value applications. Conventional approaches to biogas utilization, such as direct combustion for heat or electricity generation, fail to fully realize the energy potential of the biogas feedstock. Steam methane reforming is a known industrial process for producing hydrogen from methane-containing feedstocks, but existing reforming systems are typically designed for large-scale natural gas processing and are not well suited for integration with smaller-scale anaerobic digestion facilities or for processing biogas streams that contain significant carbon dioxide content.

Accordingly, there remains a need for integrated systems capable of efficiently converting organic waste into high-value energy products, including ultrapure hydrogen and renewable natural gas, in a manner that maximizes energy recovery and minimizes waste.

### DESCRIPTION OF THE DRAWINGS

FIG. 1 is a block diagram of an integrated waste-to-energy conversion system according to an illustrative embodiment.

FIG. 2 is the left side of a detailed schematic diagram illustrating an integrated electrically heated, waste heat recuperating, catalytic reformer system (CRS) according to an illustrative embodiment.

FIG. 3 is the right side of the diagram of FIG. 2.

FIG. 4 is a front perspective view of an illustrative SMR reactor according to an illustrative embodiment.

FIG. 5 is a side perspective view of a WGS reactor according to an illustrative embodiment.

FIG. 6 is a front perspective view of a boiler according to an illustrative embodiment.

FIG. 7 is a partial exploded perspective view illustrating internal aspects of the boiler of FIG. 6.

### DETAILED DESCRIPTION

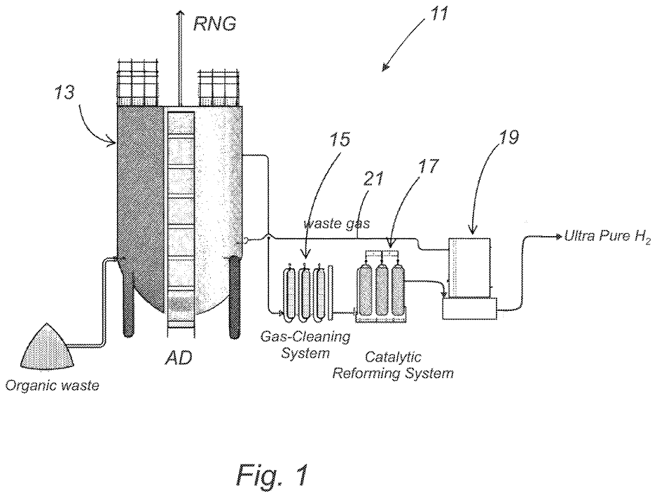

An illustrative embodiment of an integrated waste-to-energy conversion system 11 is illustrated in FIG. 1. This system 11 includes an anaerobic digester 13, a gas cleaning system 15, a catalytic reforming system (“CRS”) 17, and a hydrogen separation unit 19. The gas cleaning system 15 may also be referred to herein as gas cleaning apparatus. The CRS 17 comprises one or more catalytic reactors, as described in more detail below, and may also be referred to herein as a reforming unit. Each catalytic reactor within the CRS 17 is configured to process the cleaned biogas, or syngas derived therefrom, through catalytic reactions. The CRS 17 comprises at least one catalytic reactor that is electrically heated. The hydrogen separation unit 19 may also be referred to herein as a hydrogen separation system. The CRS 17 is fluidly connected to the anaerobic digester 13 via the gas cleaning system 15 to receive the cleaned biogas and convert it into a hydrogen-containing reformate stream (i.e., syngas). The hydrogen separation unit 19 separates the reformate stream into a hydrogen-rich product stream and a hydrogen-lean off-gas stream 21. The hydrogen separation unit 19 extracts and purifies the hydrogen from the CRS output and produces an ultra-pure hydrogen stream and a separate hydrogen-lean, carbon-containing stream. System 11 as a whole may be referred to as an integrated carbon recycling system for the selective production of biomethane and high-purity hydrogen from biogas.

The anaerobic digester 13 is designed to handle various types of organic waste, including, for example, municipal solid waste (MSW), agricultural residues, and industrial food waste. As known in the art, under anaerobic conditions, microorganisms break down the organic matter and convert gas-phase CO₂ into biomethane (biogas). The digester 13 of the illustrative embodiment is also designed to accommodate further biomethanation of a waste stream 21 generated by and received from the CRS system 17. In another embodiment, the digester 13 may be augmented by a separate anaerobic digester to further enhance the rate of biomethanation of the waste stream 21.

The raw biogas generated by the digester 13 is passed through the gas-cleaning system 15 comprising multiple units to remove trace impurities from the biogas to ensure that it is suitable for further processing in the CRS 17.

The hydrogen-rich gas mixture exiting the CRS 17 is then passed through the adsorption-based separation unit 19 that produces a high-purity hydrogen product and a separate hydrogen-lean waste stream 21 containing the carbon compounds in syngas. The waste stream 21 is fed into the anaerobic digester 13 for a biomethanation step to take place.

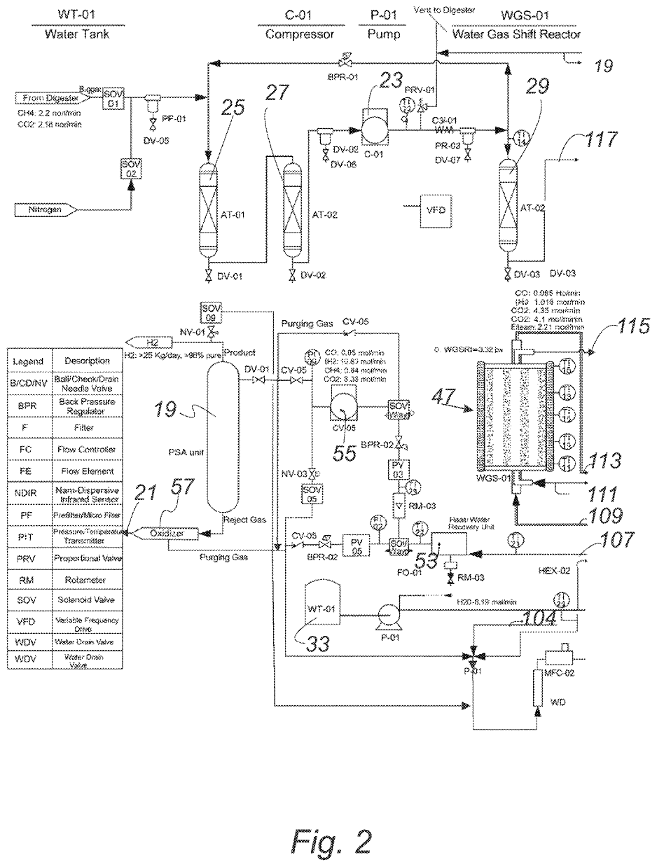

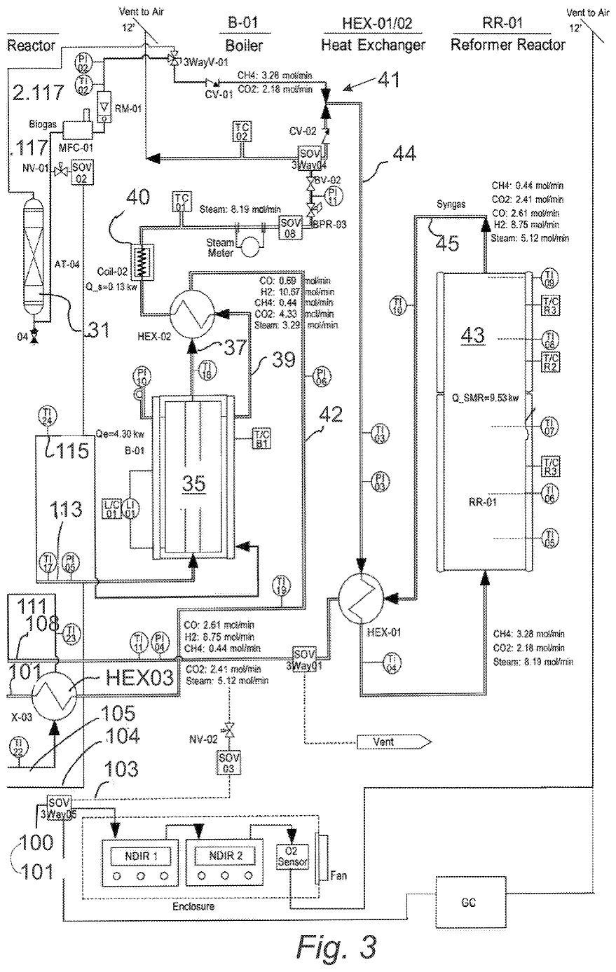

FIGS. 2 and 3 illustrate in more detail a system configured according to FIG. 1. FIG. 2 is the left-hand portion of the system, while FIG. 3 is the right-hand portion. The interconnections between the two Figures are numbered down the right-hand side of FIG. 2 and the left-hand side of FIG. 3. For example, line 101 at the bottom of FIG. 2 connects with line 101 on FIG. 3, line 103 on FIG. 2 connects with line 103 on FIG. 3, etc.

The system of FIGS. 2 and 3 includes a compressor 23 for moving the biogas input from the digester 13. In the illustrative embodiment, the gas cleaning system comprises adsorbing columns 25, 27 upstream of the compressor 23 and adsorbing columns 29, 31 downstream of the compressor 23. The upstream adsorbing columns 25, 27 may use DARCO activated carbon (AC) to remove hydrogen sulfide and silica gel (SiG) to remove water vapor before the gas is fed to the compressor 23, while the downstream columns 29, 31 may also include AC and SiG to remove remaining contaminants and water vapor after the gas has cooled. Other gas cleaning systems may be used in alternate embodiments. The Nitrogen input through SOV 02 is to purge the system at start-up and to insure there is no air left in the system.

The system of FIGS. 2 and 3 further includes a water tank 33 and a boiler 35 for generating steam containing an oxygen compound, which in the illustrative embodiment is water. The steam output 39 from the boiler 35 is passed through a heat exchanger HEX 02 and a coil 40 to a combiner 41 where it is combined with the biogas stream exiting the downstream adsorbing column 31. The coil 40 is heated using heat tapes to superheat the steam to a higher temperature. In the illustrative embodiment, the combiner 41 is a multiport valve but could be other gas mixing apparatus in other embodiments. In an illustrative embodiment, the output gas stream 44 from the combiner 41 may be: CH₄: 3.28 mol/min, CO₂: 2.18 mol/min, and Steam: 8.19 mol/min, but may be of other compositions in other embodiments. The combiner 41 functions as a mixer configured to mix the cleaned biogas with the steam output to produce a steam/biogas mixture.

The combined gas stream 44 containing methane, carbon dioxide, and steam is then passed through a heat exchanger HEX 01 and into a reformer reactor system comprising two reactors in series: (i) an electrified steam methane reforming reactor (SMR) reactor 43 containing a nickel-based supported catalyst, where the biogas/steam mixture reacts at high temperatures (>750 degrees C) to produce a syngas exit stream mixture 45 containing H₂, CO₂ and carbon monoxide (CO) together with a minor CH₄ content; (ii) a second reactor 47, which is a water gas shift (WSG) reactor containing an iron-based catalyst where the exit stream from the SMR reactor 43 further reacts at high temperatures (>300 degrees C) to convert the CO in the exit stream from SMR reactor 43 into additional H₂ and CO₂. In the illustrative embodiment, the composition of the syngas exit stream from the SMR reactor 45 may be: CO: 2.61 mol/min, H₂: 8.75 mol/min, CH₄: 0.44 mol/min, CO₂: 2.41 mol/min, Steam: 5.12 mol/min, but may be of different compositions in other embodiments. The SMR reactor 43 is a first heated reactor (e.g., electrically heated by heaters 61) that produces a syngas output comprising steam and hydrogen. The water gas shift reactor 47 is supplied with the syngas output from the SMR reactor 43 and a water input via line 111, and produces a second syngas output having an increased hydrogen concentration compared to the first syngas output, together with a heated water output via line 115. The second syngas output and the heated water output are supplied to the boiler 35, which employs the second syngas output to heat the heated water output to produce the steam output of the boiler 35. The boiler 35 outputs the second syngas output via line 37 to the hydrogen separation system 19. The SMR reactor 43 and the water gas shift reactor 47 are each a catalytic reactor within the CRS 17. The water gas shift reactor 47 is configured to employ the water input to cool it, maintaining a constant temperature at around 350-400 degrees centigrade. The CRS 17 converts the cleaned biogas into a syngas containing primarily hydrogen and carbon dioxide, and the syngas mixture may also contain CO content that is lowered by the water gas shift reactor 47.

The syngas stream 45 then passes through the heat exchanger HEX 01 and via line 109 to the water gas shift (WGS) reactor 47 to lower the CO content and increase the hydrogen content. The hot gas output 113 of the WGS reactor 47 is then passed via line 113 through the boiler 35 to heat the water inside the boiler 35 and exits the boiler 35 via line 37. In the illustrative embodiment, the composition of the hot gas output in line 113 may be: CO: 0.69 mol/min, H₂: 10.67 mol/min, CH₄: 0.44 mol/min, CO₂: 4.33 mol/min, Steam: 3.20 mol/min, but may be of other compositions in other embodiments.

The gas mixture output of the boiler 35 in line 37 is then passed through the heat exchanger HEX 03 and over line 42 to the adsorption-based separation unit 19. In the illustrative embodiment, the composition of the gas flowing in line 42 may be: CO: 0.69 mol/min, H₂: 10.67 mol/min, CH₄: 0.44 mol/min, CO₂: 4.33 mol/min, Steam: 3.20 mol/min, but may differ in different embodiments.

In the illustrative embodiment, the adsorption-based separation unit 19 is a pressure swing adsorption unit (“PSA”). Prior to input to PSA, the gas stream 42 passes through a heat recovery unit 53 and a second compressor 55. In an illustrative embodiment, the H₂ output from the PSA is 99% pure and is produced at a rate of 25 kg per day. The pressure swing adsorption unit thus produces a purified hydrogen output (i.e., ultra-pure hydrogen stream) and a separate hydrogen-lean, carbon-containing stream (waste stream 21). The hydrogen separation system 19 extracts and purifies the hydrogen from the CRS output. Other separation methods such as membrane separation may be used in alternative embodiments.

Water is pumped from the water tank 33 (FIG. 2) to supply the feedwater for the boiler 35 of FIG. 3. The water from the water tank 33 first enters the heat exchanger HEX 03 via line 105, then exits that heat exchanger and is input via line 111 into the WGS reactor 47 in order to cool the reactor 47. The water then exits the WGS reactor 47 and enters the boiler 35 via line 115.

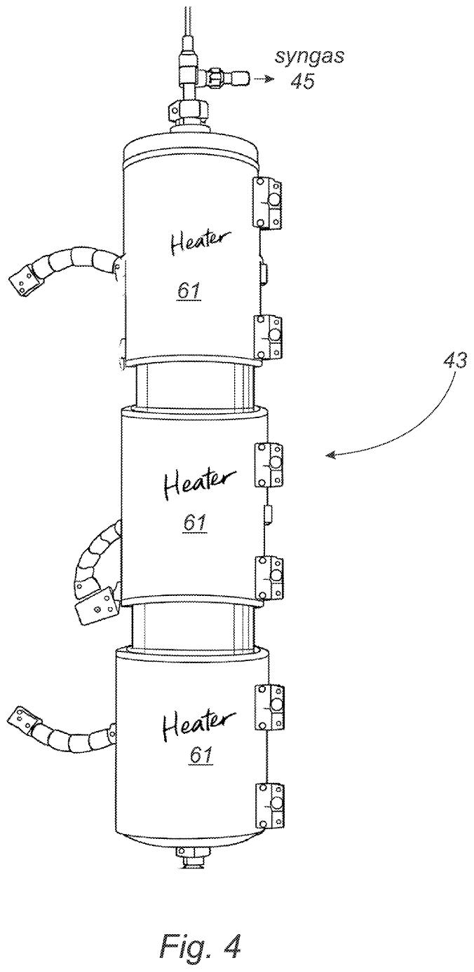

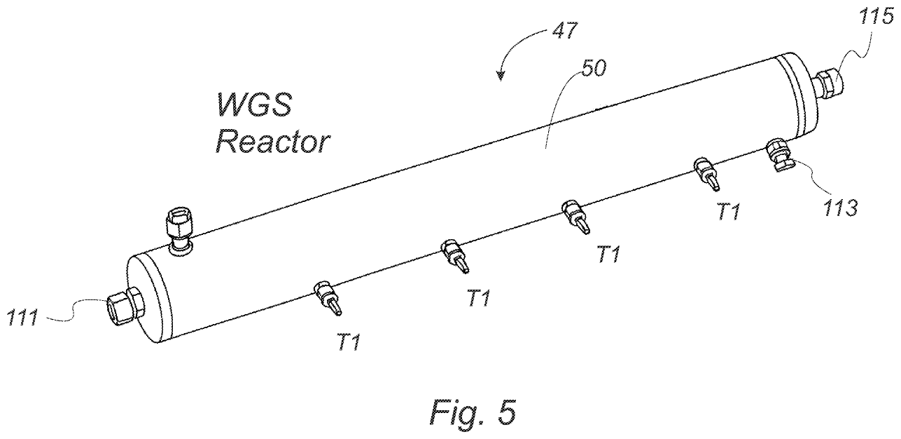

As shown in FIGS. 4 and 5, in the illustrative embodiment, the SMR reactor 43 may comprise a three-inch diameter stainless steel tube 57 with electrical heaters 61 around it, and the water gas shift (WGS) reactor 47 may be a four-inch diameter stainless steel tube 50 with a one-inch diameter tube inside it through which water passes as a cooling agent and includes temperature indicators T1 disposed along its length. In the illustrative embodiment, the WGS reactor 47 is designed to maintain high conversion with low pressure drop, and the cooling system is designed to maintain a constant temperature at around 350-400 degrees centigrade.

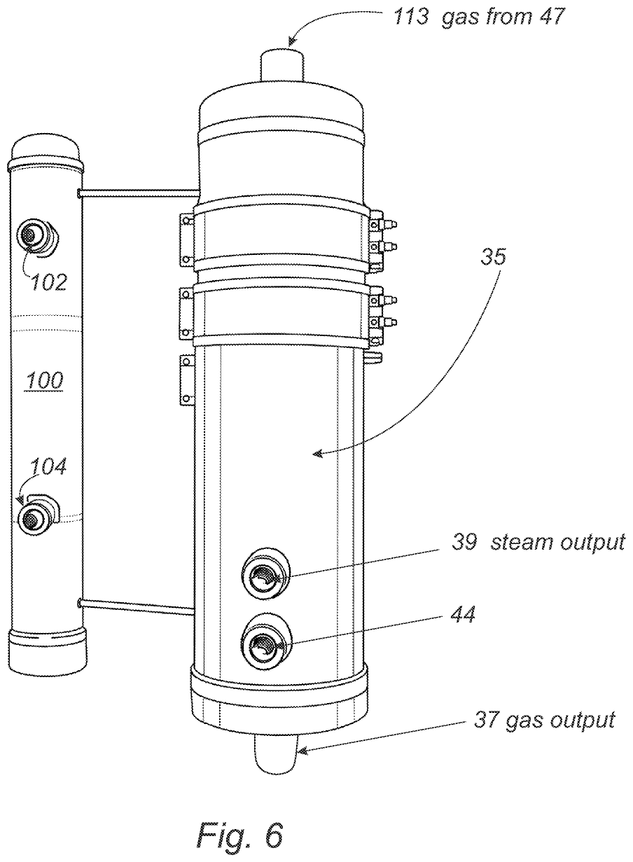

As illustrated in FIG. 6, the boiler 35 includes a central cylindrical stainless steel boiling chamber 36 with a gas input from line 113, a gas output to line 37, and a steam output to line 39. The steam input 115 is on the opposite side of the cylinder 36. A connector opening 40 accommodates a pressure relief valve. The smaller cylinder 100 includes openings 102, 104 to connect a glass level indicator.

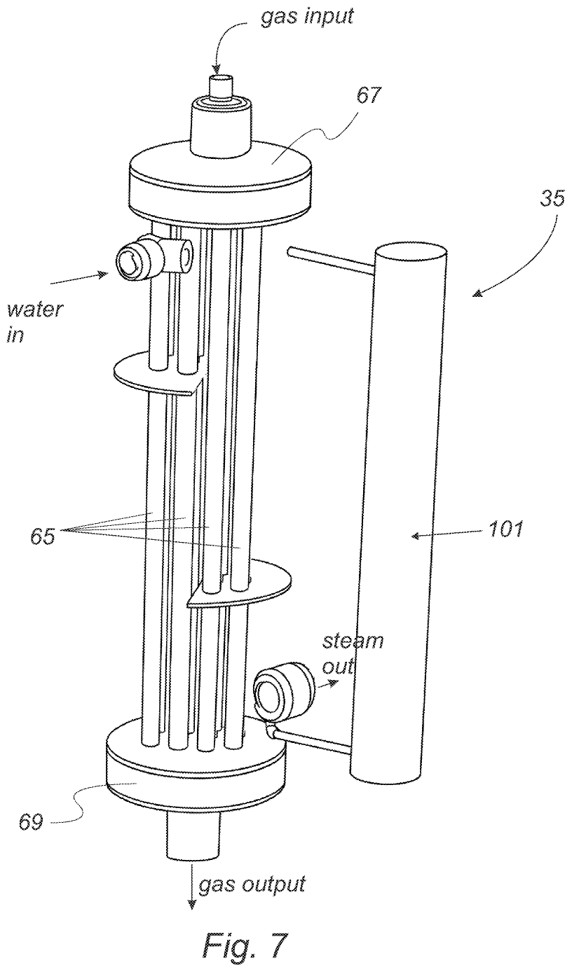

As shown in FIG. 7, internally, the boiler 35 includes a number of smaller gas transfer tubes 65 disposed between end caps 67, 69. The gas stream entering the boiler 35 is transferred via these tubes 69 to the output end cap 69 and heats the surrounding water introduced by the input line 115 to create the steam output from the boiler 35 via line 39.

In one embodiment, the PSA may be a Xebec H3300 system available from Ivys Adsorption Inc., Blainville Quebec, Canada, but could be of other configurations in other embodiments. The WGS unit 47 may employ a commercially available catalyst such as Shandong Dengzhuo Chemical Co., Ltd (DZC-F96), and the SMR reactor 43 may also employ a commercially available catalyst such as Clariant Reformermax 330.

Thus, in operation, an anaerobic digester is used to convert organic waste into biogas, followed by the processing of the biogas in an integrated electrically-heated, waste heat recuperating, catalytic reformer system (CRS) to produce ultrapure hydrogen (H₂) together with a secondary hydrogen-lean stream 21. This secondary stream undergoes further on-site biomethanation to produce renewable natural gas (RNG). Illustrative embodiments thus provide a versatile and efficient conversion of organic waste into high-valued energy products, namely RNG and H₂, through the integration of anaerobic digestion with catalytic biogas reforming. In one method of operation, at least a portion of the biogas input is diverted from the digester 13 to the CRS 17 (reforming unit), where it is converted into a hydrogen-containing reformate stream. The reformate stream is separated into the hydrogen-rich product stream and the hydrogen-lean stream containing residual hydrogen and carbon species such as CO, CO₂, and residual CH₄. The hydrogen-rich stream, or a portion thereof, may be recirculated to the digester 13 to enhance biological methanation of carbon dioxide. Biological methanation within the digester 13 converts carbon dioxide and hydrogen into methane using carbon species present in the digester environment. The method may further comprise selectively producing either high-purity hydrogen or biomethane by adjusting the relative flow rates of the hydrogen-rich stream and the hydrogen-lean stream returned to the digester 13.

Other optional equipment which may be included in various embodiments include an electricity generator such as a solar and/or wind power generation system, supplemented with an energy storage system, an electrolysis unit for further enhancing bio methanation rates, if so desired, and/or a biogas upgrading system to produce food-quality CO₂ or industrial-quality CO₂ for sale. The electrolysis unit may also be referred to as an electrolyzer, and is configured for enhancing biomethanation rates by providing additional hydrogen to the digester 13. The biogas upgrading system captures CO₂ from the biogas and produces a saleable CO₂ product of food-quality or industrial-quality grade.

In further illustrative embodiments, the system 11 is configured as a dual-purpose waste-to-energy process that can selectively and flexibly produce either methane-enriched biogas (biomethane) or ultra-pure hydrogen on demand from a common biogas feedstock through a dual recirculation carbon recycling architecture. This dual-purpose capability is achieved through the coordinated use of two recirculation pathways.

A first recirculation pathway is configured to return at least a portion of the hydrogen-rich product stream from the hydrogen separation unit 19 to the anaerobic digester 13 to enhance in-situ biomethanation. This first recirculation pathway is a hydrogen-enhanced biomethanation loop. Specifically, the recirculated hydrogen promotes the biological conversion of CO2 and H2 into methane within the digester 13 by hydrogenotrophic methanogenic archaea. The biological conversion of hydrogen and carbon dioxide into methane by hydrogenotrophic methanogenic archaea increases methane concentration in the biogas stream, enables controllable upgrading of biogas quality, and couples thermochemical hydrogen production with biological carbon conversion. The flow rate of hydrogen recirculated via this first pathway may be controlled to adjust the degree of biogas upgrading achieved within the digester 13.

A second recirculation pathway is configured to return at least a portion of the hydrogen-lean off-gas stream 21 from the hydrogen separation unit 19 (e.g., PSA reject gas) to the anaerobic digester 13, as previously described. This second pathway utilizes residual hydrogen and carbon species, including carbon-containing species (such as CO, CO₂, and residual CH₄) remaining in the off-gas stream 21 after hydrogen separation. The recirculation of this hydrogen-lean stream minimizes hydrogen loss, enhances overall carbon utilization efficiency, and enables a near CO₂-neutral, closed-loop carbon recycling system.

The coordinated operation of both the first recirculation pathway (hydrogen-rich stream) and the second recirculation pathway (hydrogen-lean off-gas stream) forms a closed-loop carbon-recycling architecture configured to selectively increase methane concentration in the biogas or to produce high-purity hydrogen on demand. The system thus enables selective production of biomethane and high-purity hydrogen from biogas. By adjusting the relative flow rates of the hydrogen-rich stream and the hydrogen-lean stream returned to the digester 13, the system 11 may be selectively operated in a biomethane enhancement mode, in which methane concentration in the biogas is increased, or a hydrogen production mode, in which high-purity hydrogen output is maximized. The system may also be operated in intermediate modes that balance biomethane and hydrogen production according to demand.

In further illustrative embodiments, the system 11, or at least the catalytic reforming system 17 (reforming unit), the hydrogen separation unit 19, the recirculation pathways, and associated control subsystems, are integrated within a modular, transportable containerized structure configured for rapid deployment and scalable expansion through addition of parallel containerized units. The containerized configuration enables rapid deployment to and integration with existing waste-to-energy or anaerobic digestion facilities. The system achieves scalable expansion through the addition of parallel containerized units to accommodate increased biogas throughput or hydrogen production demand. The modular architecture reduces installation complexity and capital expenditure compared to conventional site-built reforming installations.

In further illustrative embodiments, the system 11 includes a computer-implemented digital twin control system comprising a physics-based, hybrid digital twin platform for real-time monitoring, optimization, and control of the integrated hydrogen production and biomethanation enhancement system. The digital twin control system is configured to monitor process variables across the reforming unit, the hydrogen separation unit, and the anaerobic digester; optimize hydrogen production versus biomethane enhancement based on demand; dynamically control recirculation ratios between the hydrogen-rich product stream and the hydrogen-lean off-gas stream; predict system performance, carbon conversion efficiency, and energy balance; and enable demand-driven switching between a hydrogen-dominant operating mode and a methane-dominant operating mode. The computer-implemented digital twin control system integrates a physics-based process model representing the reforming unit, real-time data acquisition from sensors positioned throughout the system, and an optimization module configured to dynamically adjust the recirculation ratios. A control interface is configured to selectively operate the system in the hydrogen-production mode or the biomethane-enhancement mode based on outputs of the optimization module. The computer-implemented digital twin control system thus functions as an integrated control and optimization framework functionally linked to the dual recirculation carbon recycling architecture, and optimizes both carbon conversion efficiency and energy utilization across the integrated system.

### Clause Section

Unless expressly stated otherwise or technically incompatible, the features described in any one of the following paragraphs may be combined with the features described in any one or more of the other paragraphs of this clause section, whether preceding or following that paragraph. References within a paragraph to another paragraph (for example, “the apparatus described herein”) are merely illustrative and do not limit combinations.

In a first embodiment, a waste-to-energy conversion system comprises: an anaerobic digester configured to generate biogas; gas cleaning apparatus configured to remove impurities from the biogas; a catalytic reforming system (CRS) comprising at least one catalytic reactor configured to process the cleaned biogas into a syngas mixture; and a hydrogen separation system configured to separate hydrogen from the syngas mixture.

In a variation, the at least one catalytic reactor is electrically-heated.

In a further variation, the catalytic reforming system comprises a boiler configured to produce a steam output, a mixer configured to mix the biogas with the steam output to produce a steam/biogas mixture, a first heated reactor supplied with the steam/biogas mixture and configured to produce a syngas output comprising steam and hydrogen, a water gas shift reactor supplied with the syngas output and a water input and configured to produce a second syngas output comprising steam, an increased hydrogen concentration, and a heated water output, the second syngas input and the heated water output being supplied to the boiler, the boiler being configured to employ the second syngas output to heat the heated water output to produce the steam output of the boiler. As a refinement, the boiler outputs the second syngas input to the hydrogen separation system.

In a variation, the catalytic reforming system is waste heat recuperating.

In a variation, the system further includes an electrolyzer for enhancing biomethanation rates. In another variation, the system further includes a biogas upgrading system.

In a variation, the anaerobic digester processes organic waste to produce biogas. In a further variation, the CRS converts the cleaned biogas into a syngas containing primarily hydrogen and carbon dioxide.

In a variation, the hydrogen separation unit extracts and purifies the hydrogen from a CRS output and produces an ultra-pure hydrogen stream and a separate hydrogen-lean, carbon-containing stream. As a refinement, the hydrogen-lean stream is directed to the anaerobic digester for biomethanation.

In a refinement, the biogas upgrading system captures CO₂ and produces a saleable CO₂ product.

In a variation, the system further comprises a water gas shift reactor to lower the steam content in the syngas mixture. In a further variation, the hydrogen separation system comprises a pressure swing adsorption unit.

In a second embodiment, a catalytic reforming system comprises: a boiler configured to produce a steam output; a mixer configured to mix biogas with the steam output to produce a steam/biogas mixture; a first heated reactor supplied with the steam/biogas mixture and configured to produce a syngas output comprising steam and hydrogen; a water gas shift reactor supplied with the syngas output and a water input configured to produce a second syngas output comprising steam, an increased hydrogen concentration, and a heated water output; the second syngas input and heated water output being supplied to the boiler, the boiler being configured to employ the second syngas output to heat the heated water output to produce the steam output of the boiler.

In a variation, the water gas shift reactor is configured to employ the water input to cool it. In a further variation, the boiler outputs the second syngas input to a hydrogen separation system.

In a third embodiment, a method of generating hydrogen comprises: cleaning a biogas input to remove impurities from the biogas to produce cleaned biogas; employing a catalytic reforming system (CRS) comprising at least one catalytic reactor to process the cleaned biogas into a syngas mixture containing hydrogen; and employing a hydrogen separation system to separate hydrogen from the syngas mixture.

In a fourth embodiment, a method of producing hydrogen comprises: configuring a boiler to produce a steam output; mixing a biogas with the steam output to produce a steam/biogas mixture; employing a first heated reactor supplied with the steam/biogas mixture to produce a syngas output comprising steam and hydrogen; employing a water gas shift reactor supplied with the syngas output and a water input to produce a second syngas output comprising steam, an increased hydrogen concentration, and a heated water output; and further configuring the boiler to employ the second syngas output to heat the heated water output of the water gas reactor to produce the steam output of the boiler.

In a variation, the method further comprises configuring a pressure swing adsorption unit (PSA) to produce a purified hydrogen output from the second syngas input.

In a fifth embodiment, an integrated carbon recycling system for selective production of biomethane and high-purity hydrogen from biogas comprises: an anaerobic digester configured to produce biogas comprising methane, carbon dioxide, and hydrogen; a reforming unit fluidly connected to the anaerobic digester and configured to receive at least a portion of the biogas and convert the portion into a hydrogen-containing reformate stream; a hydrogen separation unit configured to separate the reformate stream into (i) a hydrogen-rich product stream and (ii) a hydrogen-lean off-gas stream; a first recirculation pathway configured to return at least a portion of the hydrogen-rich product stream to the anaerobic digester to enhance in-situ biomethanation through biological conversion of hydrogen and carbon dioxide into methane; and a second recirculation pathway configured to return at least a portion of the hydrogen-lean off-gas stream to the anaerobic digester to utilize residual hydrogen and carbon-containing species, wherein the coordinated operation of the first and second recirculation pathways forms a closed-loop carbon-recycling architecture configured to selectively increase methane concentration in the biogas or to produce high-purity hydrogen on demand.

In a variation, the first recirculation pathway is configured to promote biological conversion of CO₂ and H₂ into methane by hydrogenotrophic methanogenic archaea within the anaerobic digester.

In a variation, the system further comprises a control system configured to adjust relative flow rates of the hydrogen-rich product stream and the hydrogen-lean off-gas stream returned to the anaerobic digester to selectively operate the system in (i) a biomethane enhancement mode or (ii) a hydrogen production mode.

In a variation, the reforming unit, the hydrogen separation unit, the first and second recirculation pathways, and associated control subsystems are integrated within a modular, transportable containerized structure configured for rapid deployment and scalable expansion through addition of parallel containerized units.

In a variation, the system further comprises a computer-implemented digital twin control system comprising: a physics-based process model representing the reforming unit; real-time data acquisition from sensors positioned throughout the system; an optimization module configured to dynamically adjust recirculation ratios between the hydrogen-rich product stream and the hydrogen-lean off-gas stream; and a control interface configured to selectively operate the system in a hydrogen-production mode or a biomethane-enhancement mode, wherein the digital twin control system predicts system performance and optimizes carbon conversion efficiency and energy utilization. As a refinement, the digital twin control system is further configured to monitor process variables across the reforming unit, the hydrogen separation unit, and the anaerobic digester, and to predict carbon conversion efficiency and energy balance of the system.

In a sixth embodiment, a method for selectively producing biomethane and high-purity hydrogen from biogas comprises: generating biogas in an anaerobic digester; diverting at least a portion of the biogas to a reforming unit to produce a hydrogen-containing reformate stream; separating the reformate stream into a hydrogen-rich stream and a hydrogen-lean stream; recirculating at least a portion of the hydrogen-rich stream to the anaerobic digester to enhance biological methanation of carbon dioxide; and recirculating at least a portion of the hydrogen-lean stream to the anaerobic digester to utilize residual hydrogen and carbon species, wherein relative flow rates of the hydrogen-rich stream and the hydrogen-lean stream are adjusted to selectively operate the system in (i) a biomethane enhancement mode or (ii) a hydrogen production mode.

In a variation, recirculating at least a portion of the hydrogen-rich stream to the anaerobic digester enhances in-situ biomethanation through biological conversion of hydrogen and carbon dioxide into methane by hydrogenotrophic methanogenic archaea.

In a further variation, the method further comprises employing a digital twin control system to dynamically adjust recirculation ratios between the hydrogen-rich stream and the hydrogen-lean stream to optimize carbon conversion efficiency and energy utilization, and to enable demand-driven switching between the biomethane enhancement mode and the hydrogen production mode.

In a seventh embodiment, a computer-implemented control system for operating an integrated carbon recycling system comprising an anaerobic digester, a reforming unit, a hydrogen separation unit, and first and second recirculation pathways, the control system comprises: a physics-based process model representing the reforming unit; real-time data acquisition from sensors positioned throughout the integrated carbon recycling system; an optimization module configured to dynamically adjust recirculation ratios between (i) a hydrogen-rich product stream returned to the anaerobic digester via the first recirculation pathway and (ii) a hydrogen-lean off-gas stream returned to the anaerobic digester via the second recirculation pathway; and a control interface configured to selectively operate the integrated carbon recycling system in a hydrogen-production mode or a biomethane-enhancement mode, wherein the control system forms a digital twin that predicts system performance and optimizes carbon conversion efficiency and energy utilization.

Those skilled in the art will appreciate that various adaptations and modifications of the just described illustrative embodiments can be configured without departing from the scope and spirit of the invention. Therefore, it is to be understood that, within the scope of the appended claims, the invention may be practiced other than as specifically described herein.

What is claimed is:

1. A waste-to-energy conversion system comprising:
an anaerobic digester configured to generate biogas;
gas cleaning apparatus configured to remove impurities from the biogas;
a catalytic reforming system (CRS) comprising at least one catalytic reactor configured to process the cleaned biogas into a syngas mixture; and
a hydrogen separation system configured to separate hydrogen from the syngas mixture.

2. The system of claim 1, wherein the at least one catalytic reactor is electrically-heated.

3. The system of claim 1, wherein the catalytic reforming system comprises a boiler configured to produce a steam output, a mixer configured to mix the biogas with the steam output to produce a steam/biogas mixture, a first heated reactor supplied with the steam/biogas mixture and configured to produce a syngas output comprising steam and hydrogen, and a water gas shift reactor supplied with the syngas output and a water input and configured to produce a second syngas output comprising an increased hydrogen concentration and a heated water output, the second syngas output and the heated water output being supplied to the boiler, the boiler being configured to employ the second syngas output to heat the heated water output to produce the steam output, and the boiler further configured to output the second syngas output to the hydrogen separation system.

4. The system of claim 3, wherein the water gas shift reactor is configured to employ the water input to cool the water gas shift reactor.

5. The system of claim 1, wherein the catalytic reforming system is waste heat recuperating.

6. The system of claim 1, wherein the CRS converts the cleaned biogas into a syngas containing primarily hydrogen and carbon dioxide.

7. The system of claim 1, wherein the hydrogen separation unit extracts and purifies the hydrogen from a CRS output and produces an ultra-pure hydrogen stream and a separate hydrogen-lean, carbon-containing stream, and wherein the hydrogen-lean stream is directed to the anaerobic digester for biomethanation.

8. The system of claim 1, wherein the hydrogen separation system comprises a pressure swing adsorption unit configured to produce a purified hydrogen output from the syngas mixture.

9. A method of generating hydrogen comprising:
cleaning a biogas input to remove impurities from the biogas to produce cleaned biogas;
employing a catalytic reforming system (CRS) comprising at least one catalytic reactor to process the cleaned biogas into a syngas mixture containing hydrogen; and
employing a hydrogen separation system to separate hydrogen from the syngas mixture.

10. The method of claim 9 further comprising:
configuring a boiler to produce a steam output; mixing the cleaned biogas with the steam output to produce a steam/biogas mixture;
employing a first heated reactor supplied with the steam/biogas mixture to produce a syngas output comprising steam and hydrogen;
employing a water gas shift reactor supplied with the syngas output and a water input to produce a second syngas output comprising an increased hydrogen concentration and a heated water output;
configuring the boiler to employ the second syngas output to heat the heated water output to produce the steam output of the boiler; and
configuring a pressure swing adsorption unit to produce a purified hydrogen output from the second syngas output.

11. An integrated carbon recycling system for selective production of biomethane and high-purity hydrogen from biogas, comprising:
an anaerobic digester configured to produce biogas comprising methane, carbon dioxide, and hydrogen;
a reforming unit fluidly connected to the anaerobic digester and configured to receive at least a portion of the biogas and convert the portion into a hydrogen-containing reformate stream;
a hydrogen separation unit configured to separate the reformate stream into a hydrogen-rich product stream and a hydrogen-lean off-gas stream;
a first recirculation pathway configured to return at least a portion of the hydrogen-rich product stream to the anaerobic digester to enhance in-situ biomethanation through biological conversion of hydrogen and carbon dioxide into methane; and a
second recirculation pathway configured to return at least a portion of the hydrogen-lean off-gas stream to the anaerobic digester to utilize residual hydrogen and carbon-containing species, wherein the coordinated operation of the first and second recirculation pathways forms a closed-loop carbon-recycling architecture configured to selectively increase methane concentration in the biogas or to produce high-purity hydrogen on demand.

12. The system of claim 11, wherein the first recirculation pathway is configured to promote biological conversion of CO₂ and H₂ into methane by hydrogenotrophic methanogenic archaea within the anaerobic digester.

13. The system of claim 11 further comprising a control system configured to adjust relative flow rates of the hydrogen-rich product stream and the hydrogen-lean off-gas stream returned to the anaerobic digester to selectively operate the system in a biomethane enhancement mode or a hydrogen production mode.

14. The system of claim 11, wherein the reforming unit, the hydrogen separation unit, the first and second recirculation pathways, and associated control subsystems are integrated within a modular, transportable containerized structure configured for rapid deployment and scalable expansion through addition of parallel containerized units.

15. The system of claim 11 further comprising a computer-implemented digital twin control system comprising: a physics-based process model representing the reforming unit; real-time data acquisition from sensors positioned throughout the system; an optimization module configured to dynamically adjust recirculation ratios between the hydrogen-rich product stream and the hydrogen-lean off-gas stream; and a control interface configured to selectively operate the system in a hydrogen-production mode or a biomethane-enhancement mode, wherein the digital twin control system predicts system performance, monitors process variables across the reforming unit, the hydrogen separation unit, and the anaerobic digester, and optimizes carbon conversion efficiency and energy utilization.

16. A method for selectively producing biomethane and high-purity hydrogen from biogas, comprising: generating biogas in an anaerobic digester; diverting at least a portion of the biogas to a reforming unit to produce a hydrogen-containing reformate stream; separating the reformate stream into a hydrogen-rich stream and a hydrogen-lean stream; recirculating at least a portion of the hydrogen-rich stream to the anaerobic digester to enhance biological methanation of carbon dioxide; and recirculating at least a portion of the hydrogen-lean stream to the anaerobic digester to utilize residual hydrogen and carbon species, wherein relative flow rates of the hydrogen-rich stream and the hydrogen-lean stream are adjusted to selectively operate the system in a biomethane enhancement mode or a hydrogen production mode.

17. The method of claim 16, wherein recirculating at least a portion of the hydrogen-rich stream to the anaerobic digester enhances in-situ biomethanation through biological conversion of hydrogen and carbon dioxide into methane by hydrogenotrophic methanogenic archaea.

18. The method of claim 16, wherein the reforming unit and the hydrogen separation unit are integrated within a modular, transportable containerized structure configured for rapid deployment and scalable expansion through addition of parallel containerized units.

19. The method of claim 16 further comprising employing a digital twin control system to dynamically adjust recirculation ratios between the hydrogen-rich stream and the hydrogen-lean stream to optimize carbon conversion efficiency and energy utilization, and to enable demand-driven switching between the biomethane enhancement mode and the hydrogen production mode.

20. The method of claim 19, wherein the digital twin control system is a computer-implemented control system comprising a physics-based process model representing the reforming unit, real-time data acquisition from sensors positioned throughout the system, an optimization module configured to dynamically adjust the recirculation ratios, and a control interface configured to selectively operate the system in the hydrogen-production mode or the biomethane-enhancement mode.
